(19) United States
(12) Patent Application Publication
(10) Pub. No.: US 2020/0338042 A1
(43) Pub. Date: Oct. 29, 2020

(54) USE OF DAPHNETIN IN TREATMENT OF AGE-RELATED COGNITIVE DECLINE

(71) Applicants: XI'AN JIAOTONG UNIVERSITY, Xi'an (CN); SUZHOU YISHENGFANG BIOMEDICINE CO., LTD., Suzhou (CN)

(72) Inventors: JIAN-GANG LONG, Xi'an (CN); PEI-PEI GAO, Xi'an (CN); ZHEN WANG, Xi'an (CN); HONG-BIN QI, Xi'an (CN); YUE XIONG, Xi'an (CN); QING-QING MA, Xi'an (CN); BING LIANG, Xi'an (CN); JIAN-KANG LIU, Xi'an (CN)

(21) Appl. No.: 16/854,910

(22) Filed: Apr. 22, 2020

(30) Foreign Application Priority Data
Apr. 26, 2019 (CN) 201910342763 .7

(51) Int. Cl. A61K 31/37; A61P 25/28

(52) U.S. Cl. CPC A61K 31/37; A61P 25/28

(57) ABSTRACT

The present disclosure relates to use of daphnetin in preparation of composition for improving cognitive ability, particularly discloses use of daphnetin in preparation of a composition for preventing or treating disorders caused by increased β-amyloid level, and use of daphnetin as an inhibitor of β-amyloid. The present disclosure also relates to use of daphnetin in preparation of a composition for preventing or treating cognitive disorders, which are the Alzheimer's disease and Parkinson's disease.

### CROSS-REFERENCE TO RELATED APPLICATION

This application claims all benefits accruing under 35 U.S.C. § 119 from China Patent Application No. 201910342763.7, filed on Apr. 26, 2019 in the China National Intellectual Property Administration, the content of which is hereby incorporated by reference.

### FIELD

The present disclosure relates to the pharmaceutical field, and particularly relates to uses of daphnetin to improve cognitive ability, and in the prevention and treatment of senile dementia.

### BACKGROUND

Daphnetin, the chemical name of which is 7,8-dihydroxycoumarin, is an active ingredient extracted from Daphne Korean Nakai, and is a representative monomer of coumarins. Currently, daphnetin is clinically used in adjuvant therapy for thromboangiitis obliterans and other occlusive vascular diseases and coronary heart disease.

Alzheimer's disease (AD) is a major type of senile dementia, and is a neurodegenerative disease having the highest incidence in the elderly. AD patients account for 60-70% of the total number of dementia patients. The patients mainly manifest progressive memory loss, cognitive disorders, personality changes, and language disorders. The deposition and progressive spread of β-amyloid (Aβ) in the brain are the main signs of the series of pathophysiological changes caused by AD, and are closely related to pathological processes such as damage to mitochondrial function of nerve cells, hyperphosphorylation of tau protein, inflammatory activation of neuromuscular cells, and loss of neurons or synapses. In the early stage of AD pathogenesis, effectively inhibiting or clearing Aβ deposition and improving nerve cell metabolism are important strategies to prevent the occurrence of AD and alleviate the AD caused cognitive decline.

### SUMMARY

What is needed, therefore, is to provide a method and a composition using daphnetin for improving age-related learning and memory abilities, and particularly to provide a method of early prevention and treatment of senile dementia.

The present disclosure provides a composition for preventing or treating a cognitive disorder, the composition comprising daphnetin.

In some embodiments, the cognitive disorder is caused by an increased level of β-amyloid in a patient's brain.

In some embodiments, the disorder is the Alzheimer's disease or the Parkinson's disease.

In some embodiments, the composition further includes a pharmaceutical acceptable diluent, an excipient, or a carrier.

In some embodiments, daphnetin is the only active ingredient in the composition.

In some embodiments, daphnetin is an inhibitor in the composition, inhibiting β-amyloid in a patient's brain.

The present disclosure provides a medicine, a nutritional supplement, or cosmetics comprising the composition including daphnetin.

The present disclosure provides a method for preventing or treating a cognitive disorder, the method including administering to a patient in need thereof a therapeutically effective amount of the composition including daphnetin.

The present disclosure provides a method for preventing or treating an abnormal protein deposition, the method including contacting the abnormal protein deposition with the composition including daphnetin.

In some embodiments, the abnormal protein deposition is senile plaques.

The present disclosure provides a method for inhibiting β-amyloid in a patient's brain, the method comprising contacting a cerebral cortex of the patient with an effective amount of daphnetin.

The present disclosure provides use of daphnetin in preparing a pharmaceutical composition for preventing or treating cognitive disorder caused by an increased level of β-amyloid in a patient's brain.

The present disclosure provides use of daphnetin as an inhibitor of β-amyloid in a patient's brain.

The present disclosure provides use of daphnetin in preparing a pharmaceutical composition for preventing or treating cognitive disorders, such as the Alzheimer's disease and Parkinson's disease.

The present disclosure provides a pharmaceutical composition for reducing the level of β-amyloid, wherein daphnetin is used as the only active ingredient in the pharmaceutical composition.

The present disclosure provides a pharmaceutical composition for preventing or treating a cognitive disorder, wherein daphnetin is used as the only active ingredient in the pharmaceutical composition.

The present disclosure provides use of a composition for preventing and treating abnormal protein deposition, such as for inhibiting the formation of plaques during aging, such as senile plaques, wherein daphnetin is used as the only active ingredient in the composition.

### BRIEF DESCRIPTION OF THE DRAWINGS

Implementations are described by way of example only with reference to the attached figures.

FIG. 1A is a diagram showing the escape latencies in a water maze training for different groups of mice, wherein the abscissa represents the training day, and the ordinate represents the time for the mice to find the escape platform.

FIG. 1B is a diagram showing the path lengths in the water maze training for the different groups of mice, wherein the abscissa represents the training day, and the ordinate represents the distance for the mice to find the escape platform.

FIG. 1C is a diagram showing the areas under the curves (AUC) of the escape latencies of FIG. 1A in the water maze training for different groups of mice, wherein the abscissa represents the group of mice, and ordinate represents the area under curve of the escape latency.

FIG. 2A is a diagram showing the effect of daphnetin on reduction of the level of Aβ₄₀ in the brains of AD transgenic mice, wherein the abscissa represents the group of mice, and the ordinate represents the content of Aβ₄₀ in the cerebral cortex tissues of the mice.

FIG. 2B is a diagram showing the effect of daphnetin on reduction of the level of Aβ₄₂ in the brains of the AD transgenic mice, wherein the abscissa represents the group of mice, and the ordinate represents the content of Aβ₄₂ in the cerebral cortex tissues of the mice.

### DETAILED DESCRIPTION

A detailed description with the above drawings is made to further illustrate the present disclosure.

The inventors' recent research proved for the first time that daphnetin can ameliorate cognitive impairment and improve learning and memory abilities on mice by reducing the levels of β-amyloid (Aβ) in the brains of AD transgenic mice at the early stage of the AD pathogenesis. Daphnetin can be effective in delaying the pathogenesis and alleviating cognitive impairment at the early stage of senile dementia, such as AD, having characters such as Aβ deposition and abnormal tau phosphorylation. Daphnetin can also have an improvement effect on other protein abnormal deposition-related diseases such as the Parkinson's disease, age-related cognitive decline, and neurodegenerative changes.

The daphnetin is 7,8-dihydroxycoumarin and represented by the structural formula (I).

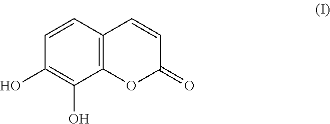 [SMILES: O=c1ccc2ccc(O)c(O)c2o1]

Daphnetin can pass through the blood-brain barrier (BBB) after being ingested, and alleviate metabolic disorders in central and peripheral tissues, and inhibit Aβ deposition in the brain, thereby improving learning and memory abilities and ameliorate cognitive impairment.

Use of daphnetin in health foods, cosmetics and drugs can prevent age-related decline of learning and memory abilities. The health foods, cosmetics and drugs can include daphnetin or daphnetin derivatives as a major effective component, or include plant extracts having daphnetin or daphnetin derivatives. The health foods, cosmetics and drugs in different dosages and forms can improve cognitive function and alleviate the decline of age-related learning and memory abilities, having an effect on early prevention of senile dementia, and alleviating cognitive disorder and plaque formation.

### Water Maze Experiment and ELISA Assay

APP/PS1 transgenic mouse model as the Alzheimer's disease transgenic mouse model was used in the experiments. The APP/PS1 double-transgenic mice overexpressing human Aβ precursor protein (APP) and presenilin 1 (PS1) in the brains can simulate typical pathological and neurological symptoms, such as Aβ deposition and cognitive function degeneration, of human AD.

A water maze experiment was used to observe the effect of daphnetin for improving the cognitive function of the mice.

The contents of Aβ₄₀ and Aβ₄₂ in the brain were detected by the enzyme-linked immunosorbent assay (ELISA) method to observe the effect of daphnetin for ameliorating abnormal protein deposition of the mice.

### 1. Experimental Materials

Daphnetin was purchased from Nanjing Puyi Biotechnology Co., Ltd.; Aβ₄₀ and Aβ₅₂ ELISA detection kits were purchased from Invitrogen Corporation.

### 2. Experimental Animal Feeding and Model Development

The 3-month-old APP/PS1 transgenic mice and control mice were purchased from the Model Animal Center of Nanjing University. All mice were fed in an environment with a temperature from 23° C. to 25° C., a humidity of 60%, and a 12 h/12 h day/night light cycles. Mice had free access to food and water during the experiment.

After adapting to the feeding environment, the experimental mice were divided into four groups: 1) a group of wild type (WT) mice to which double-distilled water (ddH₂O) was daily administered intragastrically (Group WT+ddH₂O), the number (n) of the mice is 11 (n=11); 2) a group of wild type mice to which daphnetin was daily administered intragastrically at 100 mg/kg/day (Group WT+Daphnetin), n=12; 3) a group of APP/PS1 mice to which ddH₂O was daily administered intragastrically (Group AD+ddH₂O), n=15; 4) a group of APP/PS1 mice to which daphnetin was daily administered intragastrically at 100 mg/kg/day (Group AD+Daphnetin), n=14. The entire experiment was lasted for 12 months.

### Example 1: Water Maze Experiment

The daily intragastric administrations of daphnetin or ddH₂O were lasting for 9 months to corresponding groups of mice before the water maze experiment.

Morris water maze experiment is a classic way to test spatial learning and memory abilities in mice. The experiment was conducted in a round swimming pool with a diameter of 120 cm, divided into four quadrants I, II, III, and IV. A small round platform with a diameter of 7.5 cm was placed in the center the quadrant IV, and submerged 1 cm below the water surface. Four different-shaped markers as visual cues were placed corresponding to the four quadrants on the curtain support around the swimming pool. During the experiment, the water temperature was kept at 25° C., the surroundings were kept quiet, and the swimming pool was isolated from the external environment by the curtain. Each mouse was allowed to swim freely in the swimming pool for 60 seconds (s) on the day before the formal experiment to adapt to the swimming environment. In the next 5 days, the mice were placed in different quadrants of the swimming pool. The camera placed on the top of the pool recorded the paths of the mice finding the hidden platform in the swimming pool. In the 5-days of continuous training, the mice would be artificially guided to the platform and stay on the platform for 30 s if they could not find the platform within 120 s. The times and distances that the mice travels to the platform recorded by the camera video tracking system were used to analyze the learning and memory of the mice.

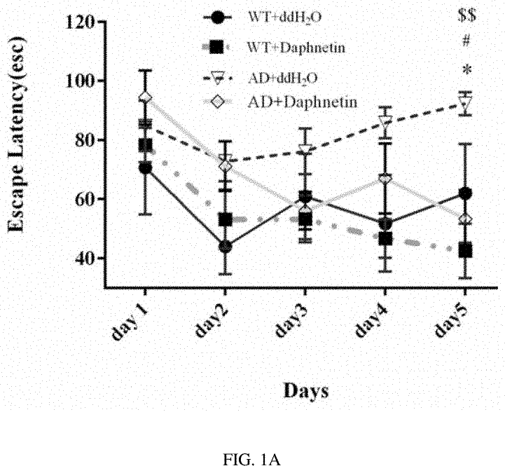

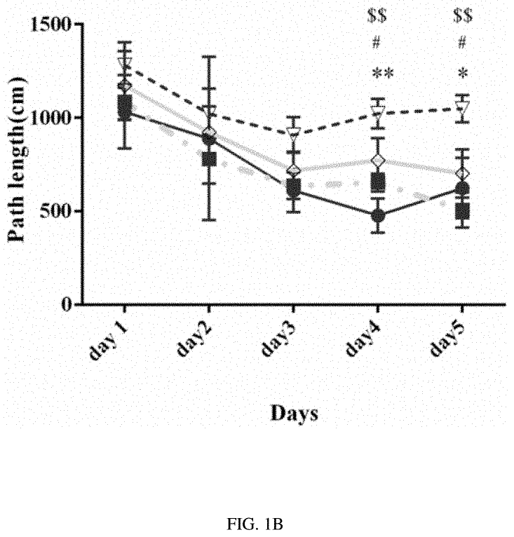

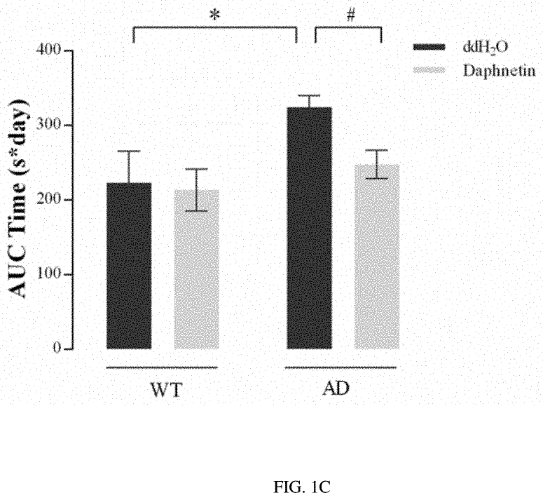

Experimental results: Referring to FIG. 1A to FIG. 1C, the results were showed in mean±S.E.M (S.E.M is standard error of mean) through statistical analysis using a Multi-way ANOVA. A statistical significance p value for WT+ddH₂O vs. AD+ddH₂O is represented by *: P<0.05, **: P<0.01, ****: p<0.0001, for AD+ddH₂O vs. AD+Daphnetin is represented by #: P<0.05, and for AD+ddH₂O vs. WT+Daphnetin is represented by $: P<0.05, $$: P<0.01.

FIG. 1A shows that on day 5, the mice in Group AD+Daphnetin had a significantly shortened latency compared to the mice in Group AD+ddH₂O in the water maze experiment. FIG. 1B shows that on day 4 and day 5, the mice in Group AD+Daphnetin has a significant shortened path length for finding the platform compared to the mice in Group AD+ddH₂O in the water maze experiment. FIG. 1C shows that the AUC of swimming time of the mice in Group AD+ddH₂O increased significantly compared to that of the mice in Group AD+Daphnetin. The above behavioral results prove that daphnetin can improve the learning and spatial memory abilities in the AD mouse model, suggesting that daphnetin can be used to alleviate or prevent cognitive decline in neurodegenerative diseases.

### Example 2: Detection of Soluble β-amyloid (Aβ) in Brain Tissue

One week after the water maze experiment in Example 1, the mice were sacrificed, and the cerebral cortex tissues of the mice were removed to detect soluble β-amyloid (Aβ) in the brain tissue.

Aβ plaque is a significant pathological feature of Alzheimer's disease. As Aβ₄₀ and Aβ₄₂ are most common in the process of producing Aβ, the contents of soluble Aβ₄₀ and Aβ₄₂ in the cerebral cortex tissues were detected by the ELISA method.

First, the cortex tissue was homogenized using a guanidine salt buffer (5 mol/L guanidine hydrochloride/50 mmol Tris HCl), and the tissue homogenate was centrifuged at 4° C. for 10 minutes at 10,000 g. The supernatant was taken for measurement. 50 μL of the known human Aβ₄₀ or Aβ₄₂ standard sample and the brain tissue homogenate sample to be tested were added to a 96-well plate pre-coated with Aβ antibody, and the plate was incubated at 37° C. for 30 minutes. Then, 50 μL of human Aβ₄₀ or Aβ₄₂ antibody detecting solution was added to each well, and the plate was incubated for 60 minutes. After aspirating the solution, the plate was washed 4 times with 1×wash buffer. After adding 100 μL of horseradish peroxidase (HRP)-labeled anti-rabbit antibody, the plate was incubated for 30 minutes. Subsequently, after aspirating the solution, the plate was washed again 4 times with 1×wash buffer. Then, 100 μL of chromogenic solution (or stabilized chromogen) was added for reaction for 30 minutes in the dark. Finally, 100 μL of stop solution was added to stop the reaction. The absorbance (OD₄₅₀) at a wavelength of 450 nm was measured by a FlexStage3 microplate reader. Protein quantification was performed by using BCA method for ELISA protein calibration.

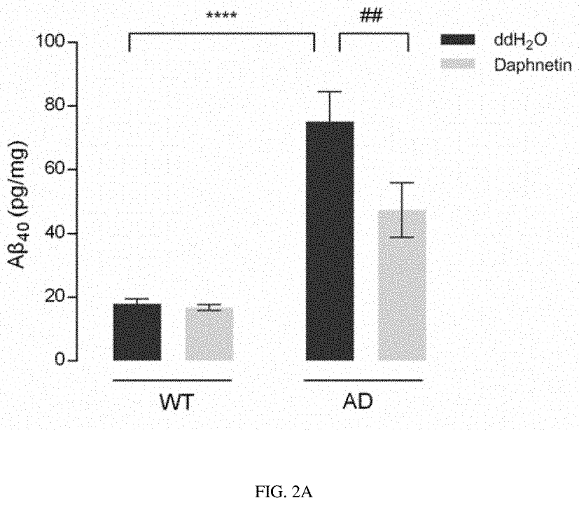

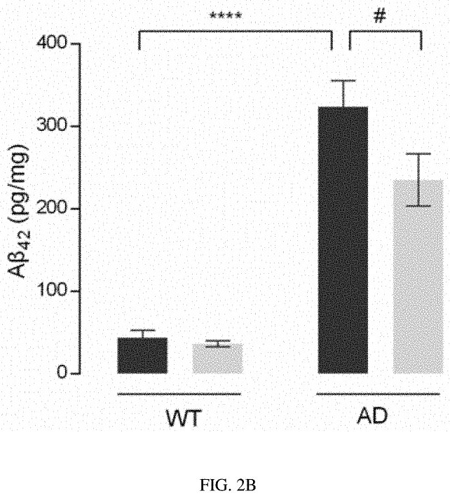

Experimental results: Referring to FIG. 2A and FIG. 2B, the results were showed in mean±S.E.M through statistical analysis using a Multi-way ANOVA. A statistical significance p value for WT+ddH₂O vs. AD+ddH₂O is represented by *: P<0.05, **: P<0.01, ****: p<0.0001; for AD+ddH₂O vs. AD+Daphnetin is represented by #: P<0.05; and for AD+ddH₂O vs. WT+Daphnetin is represented by $: P<0.05, $$: P<0.01.

FIG. 2A shows that the level of Aβ₄₀ in the cerebral cortex tissues of the mice in Group AD+Daphnetin was significantly reduced compared to that of the mice in Group AD+ddH₂O. FIG. 2B shows that the level of Aβ₄₂ in the cerebral cortex tissues of the mice in Group AD+Daphnetin was also significantly reduced compared to that of the mice in Group AD+ddH₂O. The above experimental results prove that daphnetin can reduce the levels of soluble Aβ₄₀ and Aβ₄₂ in the mouse brain and reduce the deposition of Aβ, suggesting that daphnetin ameliorates the formation of Aβ and other plaques in Alzheimer's disease and aging process.

Daphnetin, as a single-molecule compound, has been clinically used for many years. It has relatively clear pharmacological and toxicological properties. Our research proves that this compound can have new pharmacological effects, that is, to prevent and alleviate age-related cognitive function deterioration, as well as to prevent and treat senile dementia and to relieve pathological plaque deposition. This opens up new therapeutic regimens for prevention and treatment of age-related neurodegenerative diseases.

Finally, it is to be understood that the above-described embodiments are intended to illustrate rather than limit the present disclosure. Variations may be made to the embodiments without departing from the spirit of the present disclosure as claimed. Elements associated with any of the above embodiments are envisioned to be associated with any other embodiments. The above-described embodiments illustrate the scope of the present disclosure but do not restrict the scope of the present disclosure.

What is claimed is:

1. A composition for preventing or treating a cognitive disorder, the composition comprising daphnetin.

2. The composition of claim 1, wherein the cognitive disorder is caused by an increased level of β-amyloid in a patient's brain.

3. The composition of claim 1, wherein the disorder is the Alzheimer's disease or the Parkinson's disease.

4. The composition of claim 1 further comprising a pharmaceutical acceptable diluent, an excipient, or a carrier.

5. The composition of claim 1, wherein daphnetin is the only active ingredient.

6. The composition of claim 1, wherein daphnetin is an inhibitor inhibiting β-amyloid in a patient's brain.

7. A medicine, nutritional supplement, or cosmetics comprising the composition of claim 1.

8. A method for preventing or treating a cognitive disorder, the method comprising administering to a patient in need thereof a therapeutically effective amount of the composition of claim 1.

9. The method of claim 8, wherein the cognitive disorder is caused by an increased level of β-amyloid in a patient's brain.

10. The method of claim 8, wherein the disorder is the Alzheimer's disease or the Parkinson's disease.

11. A method for preventing or treating an abnormal protein deposition, the method comprising contacting the abnormal protein deposition with the composition of claim 1.

12. The method of claim 11, wherein the abnormal protein deposition is senile plaques.

13. A method for inhibiting β-amyloid in a patient's brain, the method comprising contacting a cerebral cortex of the patient with an effective amount of daphnetin.

14. Use of daphnetin in preparation of a pharmaceutical composition for preventing or treating a cognitive disorder caused by an increased level of β-amyloid in a patient's brain.
